(19) United States
(12) Patent Application Publication
(10) Pub. No.: US 2011/0144704 A1
(43) Pub. Date: Jun. 16, 2011

(54) DEVICE AND METHOD FOR ASSISTING THE ALIGNMENT OF LIMBS

(75) Inventor: Anthony Lawrence Switzer, Castle Hill (AU)

(73) Assignee: Lukemedica Pty Ltd, Blackmans Bay (AU)

(21) Appl. No.: 12/681,842

(22) PCT Filed: Oct. 7, 2008
(86) PCT No.: PCT/AU2008/001484

§ 371 Date: Feb. 28, 2011

(30) Foreign Application Priority Data
Oct. 6, 2007 (AU) 2007905484

(51) Int. Cl. A61B 17/56

(52) U.S. Cl. 606/86 R

(57) ABSTRACT

The present invention provides a device and method for determining the mechanical axis of a patient's limb. The device comprises a light source including a main light source arranged to project a beam of light onto the limb. The beam is adjusted to describe a plane of interest, to allow assessment of the mechanical axis of the limb.

### FIELD OF THE INVENTION

This invention relates to devices for assisting in the alignment of joints and limbs of the human body. The invention provides particular, but not exclusive use as a tool for medical assessment, or in the case of surgical intervention, to assist in the restoration of anatomical or functional alignment of the limb.

### BACKGROUND TO THE INVENTION

There has been an increasing interest in the replacement of damaged cartilage through the use of appropriate prosthetics and surgical intervention.

The general process for insertion of a prosthetic knee component involves the surgical resection of the damaged cartilage on the joint surfaces in the knee region together with an amount of underlying bone to provide a site for attachment of the prosthetic component. The amount of bone resection is determined by the thickness of the prosthesis and restore an appropriate amount of tension to the ligamentous structures surrounding the joint to ensure that the joint will function anatomically and transmit load applied to the joint along the mechanical axis of the limb, from the centre of the hip joint, through the centre of the knee to the ankle and foot.

An important aim for the surgeon is to ensure that the mechanical axis of the limb will be correct for the prosthesis which is to be inserted. This must be done before irreversible bone resection is carried out. Errors in attaining the correct alignment can lead to ligament imbalance and misalignment of the prosthetic components, leading to poor function of premature failure of the prosthetic device.

In order for the surgeon to be sure of the correct alignment he must be able to determine the origin of the mechanical axis, that is, the centre of the femoral/acetabular articulation, while the surgical site is remote. For example operating on the knee joint while the hip joint and the rest of the body is covered by sterile drapes necessary for the surgical procedure. X-Ray imaging to determine of the centre hip joint combined with mechanical alignment rods as a component of the knee prosthesis surgical instrumentation can be used.

The disadvantages of such an approach are considerable and include exposure of the patient and staff to greater levels of radiation from X-ray apparatus, the potential for bending of alignment rods and parallax error in the X-ray alignment. Also, the imaging process increases the operating time and increases the risk of infection. Other techniques involve the pre-operative placement of palpable radiographic markers over the femoral head which can be felt by the surgeon intra-operatively under the sterile drapes. This is highly inaccurate due to movement of the patient during surgery, skin creep, or accidental dislodgement of the marker. Preoperative imaging of the limb and measurement of the relative angles of the femoral intramedullary canal relative to the mechanical axis has been used for alignment of the femoral component, but this is limited to the femur and does not provide full limb alignment due to variables such as ligament laxity about the knee joint.

Intramedullary rod alignment also has an associated increase in the risk of complications, such as fat embolism, cortical penetration and fracture. More recently computer assisted surgical navigation has been utilized. This system uses markers attached to a frame which must be attached to the bones by surgical means such as bone pins or screws. The necessity of this attachment, increases operating time, associated surgical trauma and morbidity. Infection, haematoma and fracture at the reference frame attachment sites are published complications of this technique.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of this application.

### SUMMARY OF THE INVENTION

In a first aspect, there is provided a device for determining the alignment of a patient's limb, comprising a light source including a main light source arranged to project a beam of light onto the limb, wherein the beam is adjusted to describe a plane of interest, to allow assessment of the alignment of the limb.

The plane of interest may be the para-sagital plane transecting the centre of rotation of the hip joint of a patient.

In other words, the device may be utilised to determine a para-sagital plane transcending the centre of rotation of the hip joint. The device, in an embodiment, employs the normal movement of the hip joint and geometric triangulation by use of visible light projected on to the body in a non invasive manner. The device preferably enables the external visualization of that plane by the projection of visible light. This plane may then be used as a guide by the operator to assess the mechanical axis of the limb.

The main light source may be movable relative to the patient, and may further comprise a substantially rigid main member, arranged to receive the main light source and allow movement of the main light source along the member.

The device may also include a module for determining the relative alignment of the main light source, to allow the main light source to be aligned relative of the main light source to a surface.

The main light source may be movable along a main track located on the main member and the main track may include a geared arrangement that is arranged to move the main light source along the track. The main light source may also include a handle to facilitate movement.

The light source may include at least one additional light source, wherein the at least one additional light source is arranged to produce at least one beam of light which is parallel to the main light source beam. The at least one additional light source may contain two independent sources of light, wherein the beam of light produced by the each of the two independent sources is arranged to converge at a fixed point away from the light source.

The light source may further include a third additional light source arranged to produce a beam of light perpendicular to the main light source.

Any or all of the additional light sources may be movable to allow alignment of the additional light sources. The movement may be effected by the provision of a track, to allow the additional light sources to move along a track. The track may include a geared arrangement that is arranged to move each of the additional light sources along the track. Each additional light source may be located on a separate track.

Any or all of the additional light sources may further including handles arranged to facilitate movement of the additional light sources. The handles may be removable, for sterilisation or other purposes.

The additional light sources may be adjustable to allow alignment relative to another surface, and may also include a module for determining the relative alignment of the additional light sources. The module may be a spirit level.

The device may include an additional substantially rigid member, arranged to receive the additional light sources, which may be connected, directly or indirectly, to the main substantially rigid member. In one embodiment, the additional substantially rigid member is connected such that it is perpendicular to the main substantially rigid member to form a unit.

The unit may be connected to a counterbalance arranged to maintain the unit in a suitable orientation, and furthermore, the unit may be connected to a mounting unit, which may be adjustable to move the unit relative to a surface. The mounting unit may be connected to any one of a floor stand, a table or a ceiling.

In an embodiment, the light source is a coherent light source, such as a LASER. In one specific embodiment, the LASER is a diode LASER.

The beam of light may be projected as a line.

Each of the additional beams of light may be provided at a different frequency to facilitate the identification of each beam of light. Moreover, the main beam of light may be provided at a different frequency to any one of the additional beams of light, to facilitate the identification of the main beam of light.

In an embodiment, the light source is operated via a foot pedal.

The device may be constructed of materials suitable for sterilisation.

The device may also include a height measuring device and a radius scale arranged to allow the device to measure a coronal plane alignment.

In another embodiment, the device may include a camera arranged to facilitate documentation of a medical procedure performed with the device.

As explained above, the device may be utilised to determine a para-sagited plane transcending the centre of rotation of the hip joint. In more detail, the device employs the normal movement of the hip joint and geometric triangulation by use of visible light projected on to the body in a non invasive manner. The device then enables the external visualization of that plane by the projection of visible light. This plane can then be used as a guide by the operator to assess the mechanical axis of the limb.

In a second aspect, there is provided a method for assisting in the alignment of a patient's limb, utilising a device in accordance with a first aspect of the invention, comprising the steps of, marking a suitable point on the patient's limb, using the device to project an axial beam of light on the patient's limb, placing the patient's limb in the maximum degree of passive adduction, projecting two additional lines of light across the patient's limb until both lines converge on the suitable point, abducting the limb and adjusting the axial line of light until all lines of light align on the suitable point.

In a third aspect, there is provided a method for assisting in the alignment of a patient's limb, comprising the steps of, marking a suitable point on the patient's limb, projecting an axial beam of light on the patient's limb, placing the patient's limb in the maximum degree of passive adduction, projecting two additional lines of light across the patient's limb until both lines converge on the suitable point, abducting the limb and adjusting the axial line of light until all lines of light align on the suitable point.

### DESCRIPTION OF THE FIGURES

An embodiment, incorporating all aspects of the invention, will now be described by way of example only with reference to the accompanying drawings in which:

FIG. 1 is a figure illustrating the Anatomical Planes of the body;

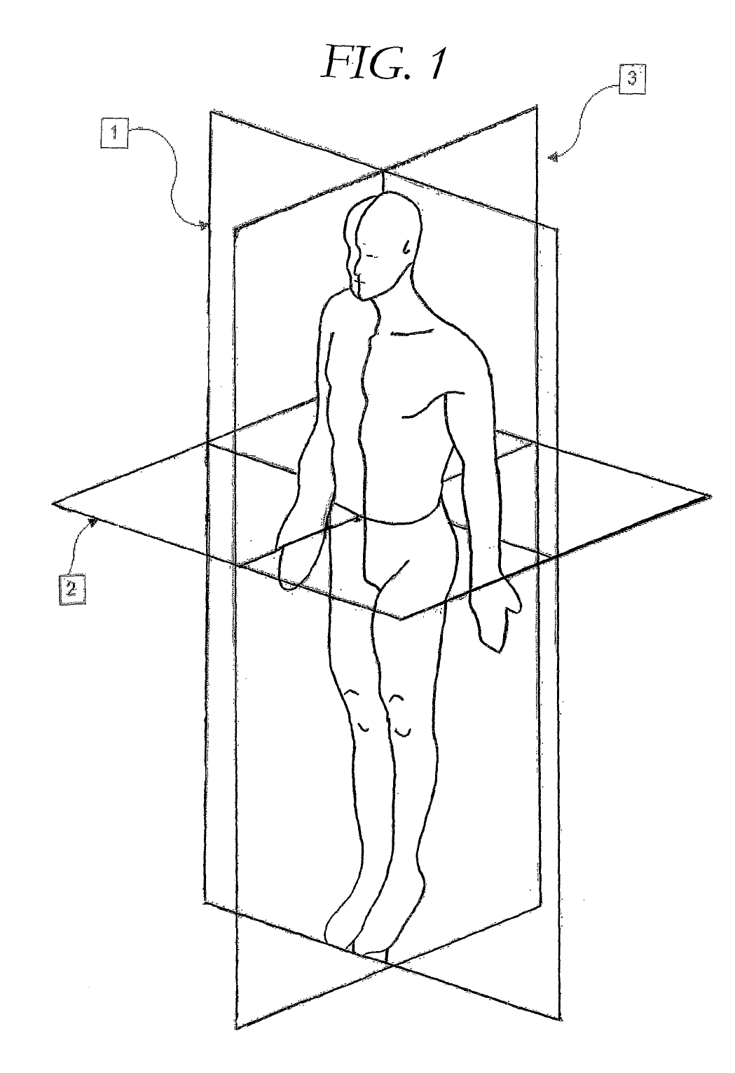

FIG. 2 is a figure illustrating the Normal Anatomic Alignment of the Lower Limb;

FIG. 3 is a figure illustrating a transverse Section of a Hip Joint;

FIG. 4 is a figure illustrating the Geometry of Motion of the Femur;

FIG. 5 is a figure illustrating an embodiment of a device positioned relative to the patient on operating; and

FIG. 6 is a figure illustrating a detailed isometric view of the Modules and Module Track of FIG. 5.

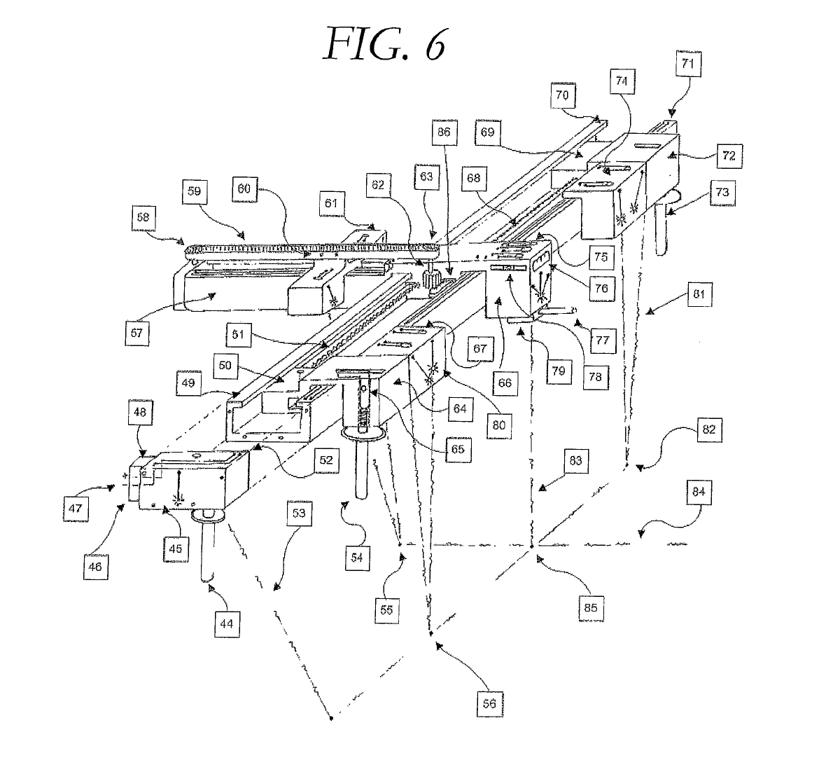

### DETAILED DESCRIPTION OF AN EMBODIMENT OF THE INVENTION

For descriptive purposes the human lower limb will be used as the exemplary application of the invention however this does not exclude application to other joints and limbs in humans or other species.

The embodiments described provide a method for determination of the centre of rotation of the joint, in this example, the centre of the head of the femur [17].

The femoral-acetabular articulation provides the origin of the weight bearing mechanical axis [4] of the lower limb.

The apparatus employs geometric triangulation and the normal anatomical movement of the hip joint, to determine the centre of the hip joint rotation [17] or planar transections through the centre of rotation. A visible light projection device, such as a diode laser line generator, is used to project this alignment in one or more planes to enable the surgeon to make corrections to the alignment of the limb.

### Geometric Basis of Function

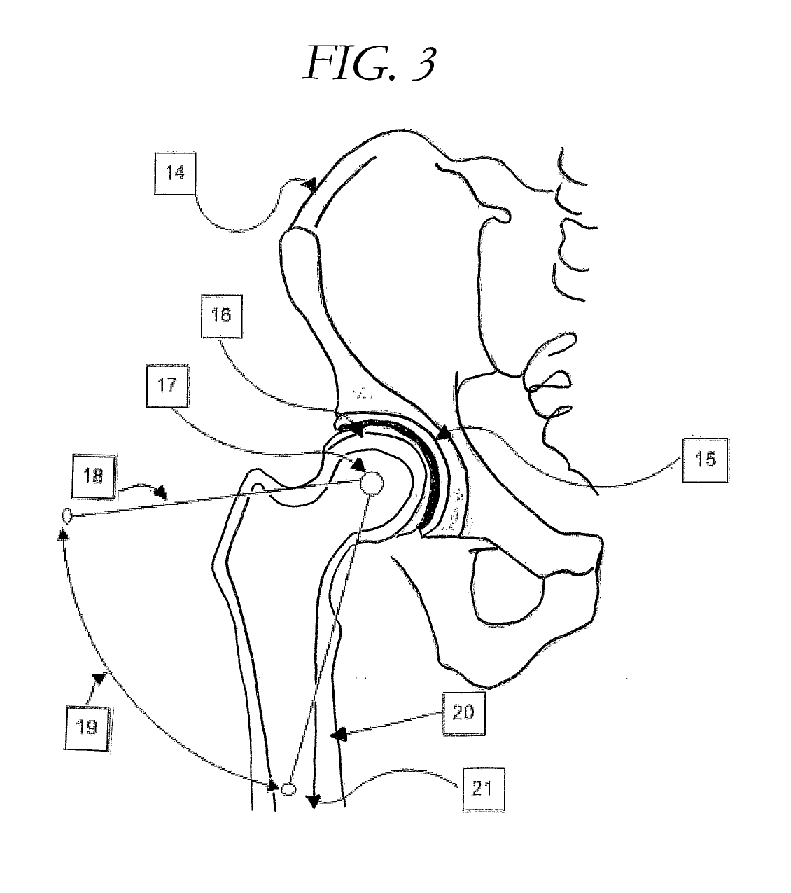

The hip, femoral-acetabular arthrosis (FIG. 3) functions effectively as a ball and socket joint. The centre of the ball is represented by a point. [17] The Femur [20], may be represented by a line [18] describing the radius of an arc of a circle [19] in two dimensions or a sphere in 3 dimensions.

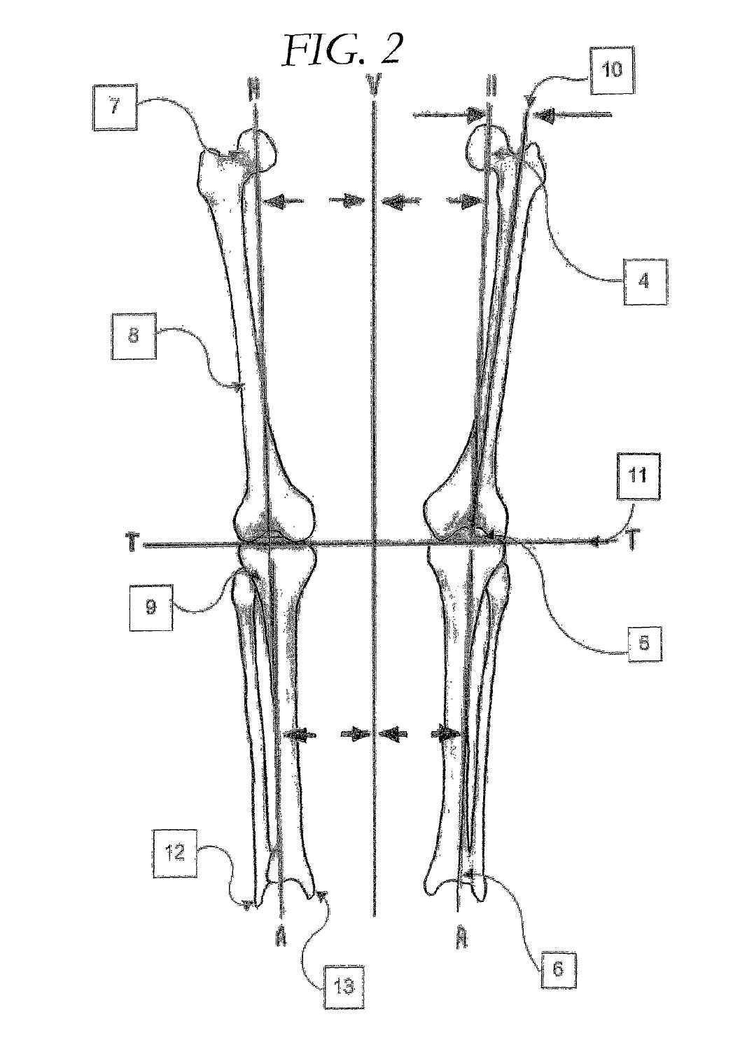

The weight bearing mechanical axis [4] of the lower limb (FIG. 2) may be represented effectively by a line passing through; the centre of the femoral head [4] the midpoint of the femoral intercondylar notch [5] of the knee and the mid maleolar point. [6]

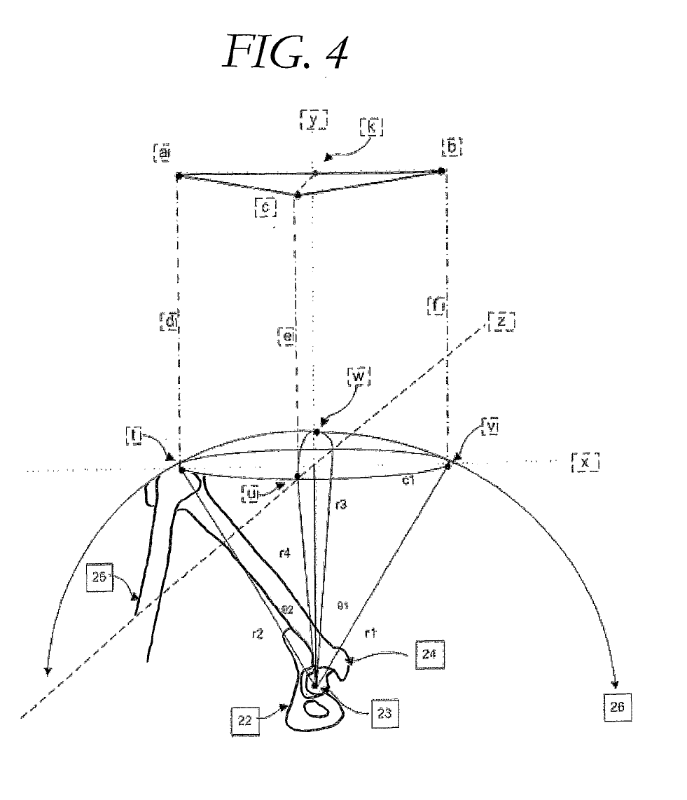

Any fixed point on the distal femur [21], when moved about the centre of rotation [17] transcribes a sector [19] of a circle if moved in one plane (FIG. 3), or a conical section of a sphere when moved in two planes. (FIG. 4). Any three points on the surface of the sphere [t] [u] [v] define a plane. (FIG. 4). The three points may also define a circle [c1] according to the circular relation x₂+y₂=r₂ where x and y are planar co-ordinates and r [r1] is the radius of the circle. A perpendicular line [y] through the centre of the circle [c1] will pass through the centre of the sphere, in this case 5 the centre of the femoral head. [23]

If three points are placed on the surface of the sphere [t] [u] [v], with known spatial separation equivalent to the diameter [t−v]/perpendicular radius [u], then a perpendicular line through the centre of the circle [y] will be concurrent with a perpendicular axis through a remote co-planar circle [a] [b] [c] separated by any known distance [d] connecting three points on its surface [a] [b] [c] by three lines of equal length [d] [e] [f] (FIG. 4). An axis of the centre of the sphere can therefore be projected from the centre of the remote circle, which may also be represented by a triangle whose base [ab]=2×the perpendicular height [yc], if the required parameters are quantifiable.

The length of the femur [r]=[23]−[t], from the center of rotation [23] to the distal reference point [t] may be calculated by measuring the height of the arc above the chord [tv] and applying the formula.

r=(m²+¼c²)/2m where c=length of the chord [tv] and m=height of the arc above the chord. (d−kw)

In an embodiment for this application the device consists of a substantially rigid main member [38] which is adjustably positional above the operating table [31]. The main member functions as a track to allow the slidable positioning of light source modules [50] placed within it. The main member may be constructed of metal, polymer or composite material and is placed perpendicular to the long axis of the body and adjustable for height and distance along the axis of the body relative to the hip joint. [38] In the preferred embodiment 5 the main member is attached to a mounting unit such as a stand [36] connected to the side mounting rail on the operating table [34]. The stand may also be free standing, roof pendant or floor mounted. The vertical component [36] of the stand is comprised of a telescoping tubular construction of sufficient resilience to prevent flexing or bending of the apparatus and of cross sectional shape to resist rotation about its axis e.g. square. The weight of the horizontal section of the device is offset by a counterweight contained in the telescoping vertical component.

The counterweight allows ease of adjustment of the vertical position by the operator. Alternatively, the weight may be offset by other means such as springs or pneumatic cylinder. The horizontal component [37] projects from the top of the vertical component perpendicular to it, along the axis of the body. The horizontal component is similarly adjustable for length by a telescoping construction. The mounting section positions the device above the sterile field to at a height convenient to the user while not interfering with the movement of the limb required for surgery or with the operating lights.

A diode laser emitter (main light source) [45], fitted with a line generator, is attached to the end of the main member (module track) [49] in such a manner as to project a line of light directly beneath the modules [74] [80] attached to the track, in the axial plane and perpendicular to the long axis of the body. The module track is also fitted modules in the form of spirit levels [78] orientated along its axis, and also perpendicular to it, to facilitate level alignment relative to a surface. Such as an operating table.

Two Laser modules (additional light sources) [74] [80] are attached to the module track in a manner that allows them to slide along the length of the resilient member. Each module contains two diode laser emitters, preferably of differing wavelength (colour) emission e.g. one red, one green. The emitters are separated by a fixed distance with the separation of the emitters being in orientation parallel to the resilient member. The emitters are fitted with line generators so as to produce lines perpendicular to the module track. The laser emitters are inclined at an angle relative to each other so that the beams coincide at preset distance below the resilient member [56] [82]. Each module is constructed so that their beams converge at the same distance. A handle [54] [73] is attached to the module to facilitate the moving of the modules along the resilient member by the user. The handles may lock the position of the module on the resilient member. The handles may be removable to allow sterilization for use in the sterile surgical field or adapted to accept sterile covers.

Each module is fitted with a bar with a gear toothed rack [51] [68] on one edge which is oriented along the length of the module track towards the module at the opposite end of the module track. The slotted bars are of sufficient length to overlap the centre point of the track and are arranged in such a manner to allow the bars to slide past each other and not impinge on the opposite module.

A projector mounting block [66] is attached to the track between the two modules. It is attached in similar manner to the modules allowing the block to slide along the track. The upper surface of the mounting block is fitted with a vertical axle [62] perpendicular to the track. This axle is fitted with a gear [62] whose teeth engage the toothed racks on the bars which are attached to the modules. The rack from one module engages one side of the gear and the rack from the other module engages on the opposite side. [86]

The construct maintains the projector mounting block in a centred position between the two modules as each or either module is adjusted for position along the track.

The projector mounting block is fitted with a perpendicular extension [57] composed of the same material and structure as the module track. The perpendicular extension is oriented towards the head [38]. The extension is fitted with a third light source in the form of a laser module [61] with two emitters similar to those attached to the resilient member. The line generators of the emitters are oriented perpendicular to those of the emitters of the modules attached to the resilient member. The end of the extension furthest from the resilient member is fitted with an axle [58] parallel to the axle on the projector mounting block. The axle is fitted with a gear [58] of similar construction as the gear on the projector mounting block. [63]

A toothed drive belt [59] to match the tooth profile of the gear is connected between the two gears with sufficient tension to maintain engagement of the teeth.

The module fitted to the extension [61] is attached to the drive belt on one side [60] in a position along its length corresponding in ratio to half of the distance of separation of the modules on the resilient member. The drive belt connection produces simultaneous movement of the module on the extension whenever the modules on the resilient member are moved and this movement is maintained in the radius/diameter ratio of the circular para-coronal plane (FIG. 4) [a] [b] [c].

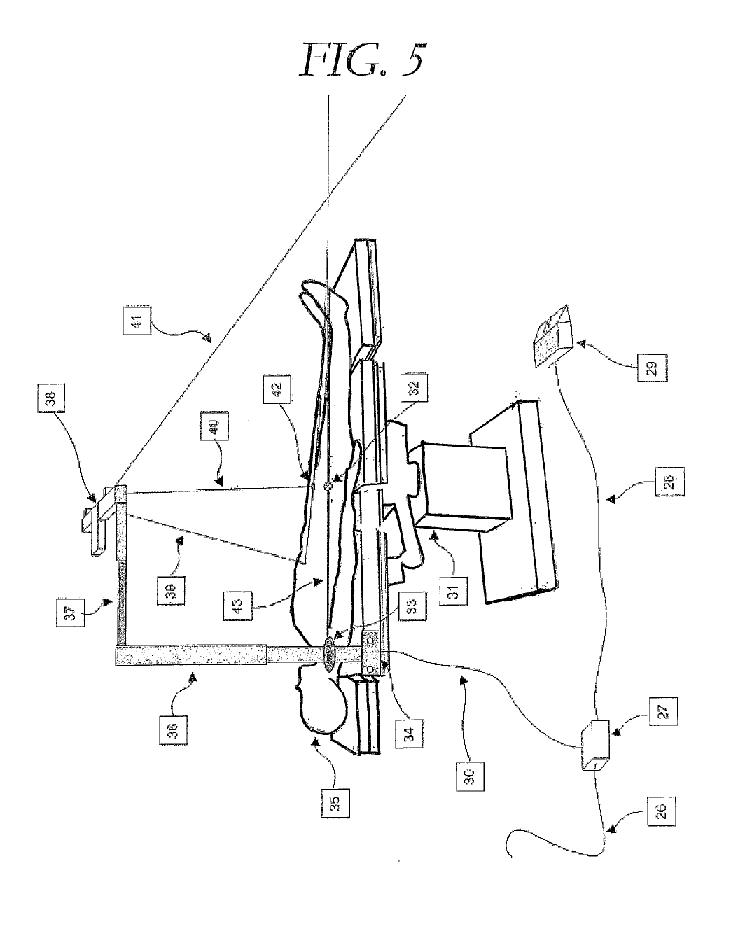

The module track maintains the projector mounting block in perpendicular alignment as the Laser modules are moved along the track. The projector mounting block [66] is fitted with a mount for a Laser line projector 5 [79]. The Laser Line Generator is mechanically fixed to the block so that its orientation maintains a projection of visible light, perpendicular to the module track and angled down from the para-coronal plane of the track in such a way as to project the line of light in a para-sagital plane [84] which can be visualised for the entire length of the limb from the hip joint to the ankle. (FIG. 5.) [42]

Each Laser module may derive its electrical power from self contained batteries or the module track may be fitted with an electrical conductor strip [47] [52] [71] to facilitate the delivery of low voltage current to the laser modules via connections which maintain contact with the strip as the modules slide on the module track. The power supply is cabled through the support stand to a battery or isolated, low voltage, supply [27].

The Laser modules electrical circuits are controlled by foot peddles switches [29] connected [28] to the power supply. This enables the surgeon/operator to activate the laser when required while leaving the hands free to attend to the procedure.

In order to assist the skilled person, a method is described that utilises the device in a total knee arthroplasty procedure.)

The device is attached to the operating table via the standard side rails on the table. (FIG. 5) [34] The attachment point is designed to 5 be remote from the surgical site and allow draping of the patient with standard sterile technique.

The patient [35] is positioned supine on the operating table and the pelvis is stabilised using a standard patient positioning device which will resist pelvic shift with movement of the leg.

The patient's hip is flexed to 90 degrees (FIG. 4). A reference point is marked on the skin of the knee over the patella or in case of surgical exposure of the knee joint, any point marked on the distal femur can be used as a reference.

With the femur approximately perpendicular to the table the laser array is activated using the foot switch [29] and the axial beam is used as a reference to adjust the position of the module track over distal femur by using adjustment handle [44]. The leg is then placed in the maximum degree of passive adduction. The most medial [74] of the laser modules is moved along the resilient member and lowered until both lines from the emitters of the module and the axial beam converge on the pre-designated point [82]=[t].

This module is then locked in position by turning the handle which activates the brake bar [65]. With the flexion of the hip maintained the leg is then abducted.

The medial-lateral position of the lateral module and the degree of abduction of the femur are adjusted until the lasers of the lateral module and the axial beam converge on the same pre-designated reference point. The convergence produces the geometric positioning of the modules and the attached alignment beam projector. The gear mechanism produces automatic centring of the alignment beam projector in a para-sagital, plane transecting the centre of rotation of the femoral head. [42]

Where the centre of rotation is required, the knee is returned 5 to the centre of the arc until the alignment beam [84] is centred on the reference mark on the distal femur. The hip is then flexed and the proximal-distal position of the module track is adjusted until the beams of the extension mounted module [61] converge on the reference point. This places the axial rotation of the alignment beam projector [83] [40] directly over the centre of rotation of the femoral head.

It will be understood that the device may also be used to describe a coronal plane. To calculate the radius of the femur (centre of rotation of the femoral head to the most distal point) the height of the arc above the geometric chord to the arc of rotation can be measured by attaching a height measuring device to the projector mounting block. The device may be composite or removably attached to the projector mounting block. The height measuring device consists of two diode laser emitters fitted with line generators contained within the projector mounting block [72]. One emitter is fixed in a plane perpendicular to the module track, the other adjustable in inclination to it and separated from it by a fixed distance. The degree of inclination is adjustable by a rotating handle [77] which may be sterilized to enable operation by the surgeon. The degree of inclination is indicated by a scale [76] calibrated by geometric triangulation to read the distance at which the laser beams converge. The radius of the femur can be then be determined by calculation as previously described. A linear scale attached to the module track is used to determine the separation pf the modules which corresponds geometrically to the length of the chord [tv] (FIG. 4).

A radius scale fitted to the vertical upright [36] of the apparatus allows the positioning of a laser line projector [33] lateral to the body in the coronal plane [1]. The vertical upright is fitted with a slotted track to enable slideable adjustment of the projector relative to the scale.

It will also be understood that the projector mounting block (or any other location on the device) may be fitted with a camera. The camera is fixed in alignment with the projected beam of the laser to allow accurate documentation of the alignment. The camera can be powered and controlled by sliding electrical contacts on the resilient member similar to those used to power the laser modules.

The embodiment is envisaged principally to provide a means of determining the mechanical axis alignment of the lower limb, intra-operatively, for the correction of deformity due to arthritis or trauma in association with the insertion of prosthetic joints in the knee. The device may also be utilised in other applications including, but not limited to, the lower and upper limb in clinical assessment, fracture reduction, osteotomy, or other surgery.

This embodiment of the invention avoids many complications associated with traditional surgical techniques by providing a means by which the surgeon/physician may determine and project the mechanical axis of the limb in the coronal [1] and sagital [3] planes in an atraumatic and non-invasive manner.

1. A device for determining the center of rotation of a hip joint of a patient, comprising:
a light source including at least one main light source arranged to project a beam of light into planes transecting the center of rotation of the hip joint,
wherein the device does not physically contact the patient.

2. A device in accordance with claim 1, wherein the main light source is movable relative to the patient.

3. A device in accordance with claim 1, further comprising a substantially rigid main member, arranged to receive the main light source.

4. A device in accordance with claim 3, wherein the main light source is movable along the substantially rigid member.

5. A device in accordance with claim 1, further comprising a device for determining the relative alignment of the main light source.

6. A device in accordance with claim 1, the main light source being adjustable to allow alignment relative of the main light source to a surface.

7. A device in accordance with claim 2, wherein the main light source is movable along the main track.

8. A device in accordance with claim 7, wherein the main track includes a geared arrangement that is arranged to move each the main light source along the main track.

9. A device in accordance with claim 1, the main light source further including a handle arranged to facilitate movement of the main light source.

10. A device in accordance with claim 1, wherein the light source includes at least one additional light source, wherein the at least one additional light source is arranged to produce at least one beam of light which is parallel to the main light source beam.

11. A device in accordance with claim 10, wherein the at least one additional light source contains two independent sources of light, wherein the beam of light produced by the each of the two independent sources is arranged to converge at a fixed point.

12. A device in accordance with claim 1, wherein the light source further includes a third additional light source arranged to produce a beam of light perpendicular to the main light source.

13. A device in accordance with claim 10, wherein the additional light sources are movable to allow alignment of the additional light sources.

14. A device in accordance with claim 13, wherein the additional light sources are each movable along a track.

15. A device in accordance with claim 14, wherein the track includes a geared arrangement that is arranged to move each of the additional light sources along the track.

16. A device in accordance with claim 14, wherein each additional light source is located on a separate track.

17. A device in accordance with any one of claim 10, the additional light sources further including handles arranged to facilitate movement of the additional light sources.

18. A device in accordance with claim 17, wherein the handles are removable.

19. A device in accordance with claim 17, wherein the handles are arranged to be sterilisable.

20. A device in accordance with claim 10, the additional light sources being adjustable to allow alignment relative to a surface.

21. A device in accordance with claim 20, further comprising a module for determining the relative alignment of the additional light sources.

22. A device in accordance with claim 21, wherein the module is a spirit level.

23. A device in accordance with claim 10, further comprising an additional substantially rigid member, arranged to receive the additional light sources.

24. A device in accordance with claim 23, wherein the additional substantially rigid member is connected to the main substantially rigid member.

25. A device in accordance with claim 24, wherein the additional substantially rigid member is connected such that it is perpendicular to the main substantially rigid member to form a unit.

26. A device in accordance with claim 25, wherein the unit is connected to a counterbalance arranged to maintain the unit in a suitable orientation.

27. A device in accordance with claim 25, wherein the unit is connected to a mounting unit.

28. A device in accordance with claim 27, wherein the mounting unit is adjustable to move the unit relative to a surface.

29. A device in accordance with claim 27, wherein the mounting unit is connectable to any one of a floor stand, a table or a ceiling.

30. A device in accordance with claim 1, wherein the light source is a coherent light source.

31. A device in accordance with claim 30, wherein the coherent light source is a LASER.

32. A device in accordance with claim 31, wherein the LASER is a diode LASER.

33. A device in accordance with claim 1, wherein the beam of light is projected as a line.

34. A device in accordance with claim 1, wherein each of the additional beams of light are provided at a different frequency to facilitate the identification of each beam of light.

35. A device in accordance with claim 34, wherein the main beam of light is provided at a different frequency to any one of the additional beams of light, to facilitate the identification of the main beam of light.

36. A device in accordance with claim 1, wherein the light source is operated via a foot pedal.

37. A device in accordance with claim 1, wherein the plane of interest is a para-sagital plane transecting the centre of rotation of the hip joint.

38. A device in accordance with claim 1, wherein the device is constructed of materials suitable for sterilisation.

39. A device in accordance with claim 1, further comprising a height measuring device and a radius scale arranged to allow the device to measure a coronal plane alignment.

40. A device in accordance with claim 1, further including a camera arranged to facilitate documentation of a medical procedure performed with the device.

41. A method for assisting in the alignment of a patient's limb, comprising the steps of, marking a suitable point on the patient's limb, projecting an axial beam of light on the patient's limb, placing the patient's limb in the maximum degree of passive adduction, projecting two additional lines of light across the patient's limb until both lines converge on the suitable point, abducting the limb and adjusting the axial line of light until all lines of light align on the suitable point.

42. A method for assisting in the alignment of a patient's limb, utilising a device in accordance with claim 1, comprising the steps of, marking a suitable point on the patient's limb, using the device to project an axial beam of light on the patient's limb, placing the patient's limb in the maximum degree of passive adduction, projecting two additional lines of light across the patient's limb until both lines converge on the suitable point, abducting the limb and adjusting the axial line of light until all lines of light align on the suitable point.
